(19) United States
(12) Patent Application Publication
(10) Pub. No.: US 2024/0225820 A1
(43) Pub. Date: Jul. 11, 2024

(54) AORTIC HEART VALVE REPLACEMENT DEVICES, SYSTEMS AND METHODS

(71) Applicant: Robert Leonardi, Lexington, SC (US)

(72) Inventor: Robert Leonardi, Lexington, SC (US)

(21) Appl. No.: 18/392,009

(22) Filed: Dec. 21, 2023

Related U.S. Application Data
(60) Provisional application No. 63/437,782, filed on Jan. 9, 2023.

(51) Int. Cl. A61F 2/24

(52) U.S. Cl. CPC A61F 2/2409; A61F 2/2418; A61F 2/243; A61F 2220/0008; A61F 2220/0025; A61F 2250/0059; A61F 2250/006; A61F 2250/0067

(57) ABSTRACT

An aortic heart valve replacement system includes a prosthetic aortic heart valve and a prosthetic aortic heart valve dock. The prosthetic aortic heart valve is moveable between a collapsed condition for directing the prosthetic aortic heart valve to the location of the patient's natural aortic heart valve and an expanded condition for fixing the prosthetic aortic heart valve at the location of the patient's natural aortic heart valve. The collapsed condition is also used for later explanting the prosthetic aortic heart valve. The prosthetic aortic heart valve dock has a ring structure configured to receive the prosthetic aortic heart valve in the expanded condition and to later release the prosthetic aortic heart valve during explantation. At least one of the prosthetic aortic heart valve or the prosthetic aortic heart valve dock includes one or more stop elements configured to prevent unwanted movement of the prosthetic aortic heart valve.

### CROSS-REFERENCE TO RELATED APPLICATION

This application claims the benefit of U.S. Provisional Patent Application Ser. No. 63/437,782, filed Jan. 9, 2023 (pending), the disclosure of which is incorporated by reference herein in its entirety.

### TECHNICAL FIELD

The present disclosure generally relates to devices, systems and methods for aortic heart valve replacement.

### BACKGROUND

Aortic stenosis is the most common form of aortic valve disease, affecting more than 1.5 million Americans (more than 500,000 with severe disease) according to the American Heart Association. Patients with severe aortic stenosis are treated with aortic valve replacement (AVR), and roughly 100,000 AVR procedures (AVRs) are performed annually in the United States alone. Presently all AVRs are completed with permanent prosthetic aortic heart valve implants. Some AVRs are completed with mechanical valves, which are considered permanent solutions because they do not deteriorate. However, mechanical valves require long-term treatment with anticoagulant medications. Therefore, most AVRs are completed with bioprosthetic valves, which generally do not require long-term treatment with anticoagulant medications. However, bioprosthetic valves are not considered permanent solutions because they inevitably deteriorate, which can lead to significant clinical issues 10 to 15 years after implantation and, too often, even sooner.

Patients undergoing bioprosthetic AVR therefore often face the challenges of needing another AVR a number of years after the initial AVR, and that the second AVR may be more complicated than the first. Much of the additional complexity with the second and subsequent AVRs is related to the presence of a failed valve that was implanted initially in a “permanent” manner, i.e., a manner that makes the replacement procedure difficult and riskier. Patients undergoing a procedure to replace a prosthetic aortic heart valve are also often poor candidates for surgical explantation of the previously implanted prosthetic aortic heart valve. Often, such an AVR procedure is not an appealing option even for good candidates. On the other hand, leaving the existing prosthetic aortic heart valve in place leaves chronic and, typically, increasing challenges for both the patient and healthcare providers.

Presently available prosthetic aortic heart valves are designed for permanent implantation either surgically by open-heart surgery (SAVR) or percutaneously by transcatheter AVR (TAVR). SAVRs may be mechanical or bioprosthetic. TAVRs are generally all bioprosthetic. Mechanical aortic valve prostheses require permanent anticoagulation therapy, which decreases thrombotic risk at the expense of increased bleeding risk. Over the years following SAVR with a mechanical valve, bleeding events are seemingly inevitable. Bleeding events require interruption of anticoagulation therapy, and this increases risk of mechanical valve thrombosis. Mechanical valve thrombosis may lead to valve dysfunction, which can then require treatment with risky thrombolytic drugs and/or surgical explantation and replacement of the dysfunctional mechanical valve. Also, mechanical valve thrombosis may lead to systemic arterial thrombo-embolism with consequent stroke or other, life-threatening ischemia/infarction. After interruption of anticoagulation therapy, when sources of bleeding are controlled and bleeding risk is thought to again be acceptable, anticoagulation therapy is resumed. Thrombotic risk is once again exchanged for bleeding risk.

Bioprosthetic aortic valves all degenerate and would ideally be replaced during the life of the patient. Therefore, many patients undergoing AVR with a bioprosthesis will eventually need a replacement or “redo” AVR. Some patients may need a third or even a fourth redo AVR. For example, a 40-year-old patient could easily need four bioprostheses to survive to age 80. A 40-year-old patient may need more than four bioprostheses to reach age 80 because younger patients tend to “wear out” bioprostheses more quickly than older patients. For example, the first prosthetic aortic valve or two may last less than 10 years or even less than five years. Transcatheter bioprostheses also fail, probably at a rate like surgical bioprostheses, although this is an area of ongoing research.

Presently, there are two options for replacement of dysfunctional surgical and transcatheter aortic valve bioprostheses. These options are surgical redo AVR, in which the existing prosthetic valve is surgically removed before a new prosthetic valve is sewn into place, and “valve-in-valve” AVR, in which a transcatheter valve is deployed inside the existing SAVR or TAVR. With this latter option, the existing prosthetic aortic heart valve is not removed. Surgical redo AVR is highly invasive in every case, is usually riskier than the first AVR, and is often avoided in favor of valve-in-valve AVR or no replacement AVR at all.

With a valve-in-valve AVR, the existing bioprosthetic leaflets are permanently fixed in the “open” position when the new (replacement) prosthetic aortic heart valve is expanded and thereby implanted within the existing valve. In most cases, this results in some limitation of access to the coronary arteries during future catheterization procedures, which can be a significant problem for patients with coronary artery disease. In the worst case this leads to fatal myocardial infarction. Also, effective orifice area and, therefore, the ease of blood flow through the valve, is diminished with every valve-in-valve AVR. In this regard the existing prosthetic valve(s) take up space within the natural aortic valve annulus, and patient-prosthesis mismatch (where the normally functioning new valve is too small for the patient) becomes a bigger problem.

For reasons such as those given above, many patients with dysfunctional aortic valve bioprostheses are anatomically poor candidates for valve-in-valve AVR and have limited options. Some of these patients will undergo relatively invasive and risky surgical redo AVR procedures. Others will undergo relatively complex valve-in-valve AVRs, including additional procedures to mitigate risk of coronary artery occlusion and patient-prosthesis mismatch. Coronary artery occlusion risk may be mitigated by Bioprosthetic Aortic Scallop Intentional Laceration to prevent Iatrogenic Coronary Artery obstruction (BASILICA) or by “snorkel stenting.” Patient-prosthesis mismatch may be reduced by intentional fracturing and expansion of an old bioprosthetic SAVR, but fracturing is not really an option for older TAVRs.

Because of risks related to redo AVR, some patients with failed aortic valve bioprostheses will choose to accept low quality of life and a poor prognosis for survival. Many of today's patients do not choose mechanical prosthetic aortic valves because of the long-term bleeding risk, and they don't want bioprosthetic valves because of the specter of increasingly complex redo AVRs every 10 to 15 years (or more frequently) in the future. Because of the limitations of current AVR technologies, “lifetime management” of aortic valve disease has become an increasingly studied subject in cardiovascular research.

For these and other reasons, it would be desirable to provide improved manners of replacing previously implanted prosthetic aortic heart valves.

### SUMMARY

In one illustrative embodiment, an aortic heart valve replacement system is provided and includes a prosthetic aortic heart valve movable between a collapsed condition for directing the prosthetic aortic heart valve to the location of the natural aortic heart valve and an expanded condition for fixing the prosthetic aortic heart valve at the location of the natural aortic heart valve of a patient. The collapsed condition is also used for later explanting the prosthetic aortic heart valve. Of course, it is to be understood that during explantation the prosthetic aortic heart valve may be more or less “collapsed” than when it was originally implanted. A prosthetic aortic heart valve dock is included in the system and has a ring structure configured to receive the prosthetic aortic heart valve in the expanded condition and to release the prosthetic aortic heart valve during an explantation procedure when the prosthetic aortic heart valve needs to be replaced. At least one of the prosthetic aortic heart valve or the prosthetic aortic heart valve dock includes one or more arresting elements (such as, for example, frictional surface elements and/or at least one stop element in the form of a projecting portion) configured to prevent unwanted movement of the prosthetic aortic heart valve within the dock. It will be appreciated that other forms of arresting elements may be used in addition to or instead of those more specifically discussed herein.

The system may include one or more additional, optional, and/or more specific features. The prosthetic aortic heart valve dock may include a sewing ring portion configured to be surgically implanted by suturing the sewing ring portion to aortic tissue at the location of the natural aortic heart valve. Alternatively, the prosthetic aortic heart valve dock may be configured to be implanted using a transcatheter procedure. At least one of the prosthetic aortic heart valve dock or the prosthetic aortic heart valve may include at least one frictional surface element. Instead of or in addition to frictional surface elements, at least one of the prosthetic aortic heart valve dock or the prosthetic aortic heart valve may include at least one projecting portion or “stop element.” The stop element may be directed radially inward along the prosthetic aortic heart valve dock or radially outward along the prosthetic aortic heart valve. As mentioned, other forms of arresting elements may be used in addition to or instead of frictional elements, stop elements or any other element having a similar function.

In another illustrative embodiment, an aortic heart valve replacement system includes a prosthetic aortic heart valve movable between a collapsed condition for directing the prosthetic aortic heart valve to the location of the natural aortic heart valve and an expanded condition for fixing the prosthetic aortic heart valve at the location of the natural aortic heart valve of a patient. The collapsed condition is also used for later explanting the prosthetic aortic heart valve, such as discussed herein. A prosthetic aortic heart valve dock is included in the system and has a ring structure configured to receive the prosthetic aortic heart valve in the expanded condition and configured to release the prosthetic aortic heart valve during an explantation procedure when the prosthetic aortic heart valve needs to be replaced. At least one of the prosthetic aortic heart valve or the prosthetic aortic heart valve dock may include features and/or structures that inhibit future endothelial growth and/or facilitate the destruction of endothelial growth between the prosthetic aortic heart valve and the prosthetic aortic heart valve dock at least to an extent that loosens adhesion between these two elements.

The aortic heart valve replacement system may have one or more additional, optional, and/or more specific features. For example, inhibiting endothelial growth may include a drug-eluting coating or treatment on at least one of the contacting surfaces of the prosthetic aortic heart valve or the prosthetic aortic heart valve dock. The drug may be any medication designed to inhibit endothelial growth. A radially outward facing surface portion of the prosthetic aortic heart valve may include the drug-eluting coating. A radially inward facing surface portion of the prosthetic aortic heart valve dock may include the drug-eluting coating. Destroying endothelial growth may include the use of at least one electrically conductive element configured to receive a contacting conductor of an electrosurgical element to facilitate the transfer of electrical energy to the interface between the prosthetic aortic heart valve and the prosthetic aortic heart valve dock sufficient to at least assist with disengagement of existing endothelial growth and attendant adhesion between the prosthetic aortic heart valve and the prosthetic aortic heart valve dock. The electrically conductive element may include an electrical terminal or terminals extending from the prosthetic aortic heart valve dock or the prosthetic aortic heart valve and configured to be engaged by an electrically conductive engaging device, such as a snare, endovascular forceps, valve explantation device, or similar device.

Another illustrative embodiment of an aortic heart valve replacement system may include a prosthetic aortic heart valve movable between a collapsed condition for directing the prosthetic aortic heart valve to the location of the natural aortic heart valve and an expanded condition for fixing the prosthetic aortic heart valve at the location of the natural aortic heart valve of a patient. The collapsed condition is also used for later explanting the prosthetic aortic heart valve, such as discussed herein. A prosthetic aortic heart valve dock is included in the system and has a ring structure configured to receive the prosthetic aortic heart valve in the expanded condition and configured to release the prosthetic aortic heart valve during an explantation procedure when the prosthetic aortic heart valve needs to be replaced.

Another illustrative embodiment of an aortic heart valve replacement system includes a prosthetic aortic heart valve movable between a collapsed condition for directing the prosthetic aortic heart valve to the location of the natural aortic heart valve and an expanded condition for fixing the prosthetic aortic heart valve at the location of the natural aortic heart valve of a patient. The collapsed condition is also used for later explanting the prosthetic aortic heart valve, such as discussed herein. A prosthetic aortic heart valve explantation device is included in the system and has a portion movable between an expanded condition configured to engage the prosthetic aortic heart valve and a collapsed condition configured to be directed within a catheter. The prosthetic aortic heart valve includes engageable elements capable of being grasped or otherwise engaged and held by the portion of the explantation device allowing the prosthetic aortic heart valve to be pulled into the catheter.

The system may have one or more additional, optional, and/or more specific features. For example, the engageable elements may include hook-like elements. Instead or in addition, the engageable elements may be part of a radially expandable and collapsible stent structure of the prosthetic aortic heart valve. The explantation device may include hook-like elements configured to engage with the prosthetic aortic heart valve. In other embodiments the explantation device may include a snare-like structure. The snare-like structure may have one or more loops that could constrict or collapse and engage with suitable structure on the prosthetic aortic heart valve for purposes of pulling on and extracting the prosthetic aortic heart valve from a prosthetic aortic heart valve dock during a transcatheter explantation process. The explantation device may involve a snare or set of snares that engage elements of the prosthetic aortic heart valve. The explantation device may engage with the stent structure of the prosthetic aortic heart valve. The explantation device may engage with the prosthetic aortic heart valve by expanding in a radially outward direction. Alternatively, the explantation device may engage with the prosthetic aortic heart valve by collapsing in a radially inward direction. The explantation device may include a steerable catheter and/or a catheter having a bent distal tip portion.

In another illustrative aspect, a prosthetic aortic heart valve replacement method is provided and includes directing a catheter to the location of the natural aortic heart valve where a prosthetic aortic heart valve was previously implanted within a prosthetic aortic heart valve dock. The prosthetic aortic heart valve dock may have been surgically implanted by suturing the sewing ring portion to aortic tissue at the location of the natural aortic heart valve or may have been implanted using a transcatheter procedure. The prosthetic aortic heart valve may have been secured in the prosthetic aortic heart valve dock using one or more arresting elements configured to prevent unwanted movement of the prosthetic aortic heart valve within the prosthetic aortic heart valve dock. At the time of replacement of the previously implanted or “old” prosthetic aortic heart valve, the old prosthetic aortic heart valve is removed from the prosthetic aortic heart valve dock, which remains in place at the location of the natural aortic heart valve. Removal of the old prosthetic aortic heart valve from the prosthetic aortic heart valve dock is facilitated by collapsing the old prosthetic aortic heart valve into the distal end of an explantation catheter. Once collapsed into the distal end of the explantation catheter and removed from the prosthetic aortic valve dock, the old prosthetic aortic heart valve may be removed from the patient using the explantation catheter. A new prosthetic aortic heart valve may then be implanted into the prosthetic aortic heart valve dock using a transcatheter procedure. The method may include various additional, optional, and/or more specific methodology and/or involve the use of various additional, optional, and/or more specific structures. Adhesion caused by bonding endothelial growth between the prosthetic aortic heart valve dock and the prosthetic aortic heart valve may be released or, in other words, loosened to facilitate explantation of the old prosthetic aortic heart valve. Loosening adhesion caused by bonding endothelial growth between the prosthetic aortic heart valve dock and the prosthetic aortic heart valve may involve establishing an electrical connection with at least one of the prosthetic aortic heart valve dock and/or the prosthetic aortic heart valve to facilitate the transfer of electrosurgical energy to the interface between the prosthetic aortic heart valve dock and the prosthetic aortic heart valve sufficient to assist with disruption of the bonding endothelial growth. Establishing the electrical connection may include engaging an electrical terminal extending from at least one of the prosthetic aortic heart valve dock and/or the prosthetic aortic heart valve. Establishing the electrical connection may also include engaging the prosthetic aortic heart valve itself. Loosening adhesion caused by bonding endothelial growth may involve the application of electrosurgical energy to at least one of the prosthetic aortic heart valve dock or the prosthetic aortic heart valve. For example, electrosurgical energy may be applied in a bipolar arrangement (with both electrodes residing inside the patient) to localize energy application at the dock-valve interface, minimizing energy loss and non-target tissue injury during transcatheter electrosurgical disruption of bonding endothelial growth. The electrosurgical energy may be supplied by an electrosurgical generator or any device that applies energy in a safe and effective manner for loosening adhesion between the prosthetic aortic heart valve dock and the prosthetic aortic heart valve.

Removing the prosthetic aortic heart valve from the prosthetic aortic heart valve dock may include grasping or otherwise engaging and holding engageable elements of the prosthetic aortic heart valve with an explantation device and pulling the prosthetic aortic heart valve into the catheter. The explantation device may include hook-like elements, and removing the prosthetic aortic heart valve may include grasping the prosthetic aortic heart valve with the hook-like elements. The explantation device may include a stent-like structure, and removing the prosthetic aortic heart valve may include grasping or otherwise engaging and holding the prosthetic aortic heart valve with the stent-like structure. Grasping or otherwise engaging and holding the prosthetic aortic heart valve may include expanding the stent-like structure in a radially outward direction or collapsing the stent-like structure in a radially inward direction. Directing the catheter may include directing a steerable catheter and/or a catheter with a bent distal tip portion to the location of the natural aortic heart valve where a prosthetic aortic heart valve was previously implanted.

Additional features and advantages of the inventive aspects will become more apparent upon review of the following detailed description taken together with accompanying drawings of the illustrative and exemplary embodiments.

### BRIEF DESCRIPTION OF DRAWINGS

FIG. 1 is a schematic view of a patient and the anatomy of the heart.

FIG. 2 is a schematic, enlarged view showing a portion of the heart and, specifically, a stenotic natural aortic valve in cross-section.

FIG. 3 is a perspective view of a surgically implantable prosthetic aortic heart valve dock in accordance with one illustrative embodiment of the invention.

FIG. 3A is an enlarged portion of the dock shown in FIG. 3.

FIG. 3B is an enlarged cross-sectional view of a portion of the dock shown in FIG. 3A illustrating an illustrative embodiment of a stop element.

FIG. 4 is a cross-sectional view of a prosthetic aortic heart valve in accordance with an illustrative embodiment of the invention.

FIG. 5A is a schematic view illustrating an initial portion of a method of implanting a prosthetic aortic heart valve in accordance with an illustrative embodiment of the invention.

FIG. 5B is a schematic view similar to FIG. 5A but illustrating a later portion of the implantation process after which the prosthetic aortic heart valve has been expanded into the dock.

FIG. 6A illustrates the snaring of an electrically conductive terminal during the explantation procedure.

FIGS. 6B and 6C illustrate subsequent steps during which electrosurgical energy is applied to loosen adhesion caused by, for example, endothelial growth between the prosthetic aortic heart valve dock and the prosthetic heart valve.

FIGS. 6D through 6G illustrate steps during which an explantation device is used to grasp, collapse, and remove the prosthetic aortic heart valve from the prosthetic aortic heart valve dock using an explantation catheter and, possibly, the application of electrosurgical energy to loosen adhesion caused by, for example, bonding endothelial growth between the prosthetic aortic heart valve dock and the prosthetic aortic heart valve.

FIG. 6H illustrates a subsequent step of implanting a replacement prosthetic aortic valve into the prosthetic aortic valve dock.

FIG. 7A is an elevational view of an explantation device having a stent-like structure with hooks that can engage a prosthetic aortic heart valve upon expansion.

FIG. 7B illustrates the explantation device of FIG. 7A in a collapsed condition within a catheter.

FIG. 8A is a partially sectioned elevational view illustrating another illustrative embodiment having a combined prosthetic aortic heart valve and a prosthetic aortic heart valve transcatheter dock that are implanted during a transcatheter procedure.

FIG. 8B is a schematic view of the methodology used during the transcatheter procedure to insert the not-yet-expanded prosthetic aortic heart valve and prosthetic aortic heart valve transcatheter dock into a natural aortic heart valve.

FIG. 8C is a schematic view illustrating the implanted, combined prosthetic aortic heart valve and prosthetic aortic heart valve transcatheter dock within the natural aortic heart valve.

FIG. 8D illustrates the prosthetic aortic heart valve being explanted from the prosthetic aortic heart valve transcatheter dock by collapsing the prosthetic aortic heart valve into an explantation catheter.

FIG. 9 is an elevational view illustrating another alternative embodiment of a prosthetic aortic heart valve and a prosthetic aortic heart valve transcatheter dock.

FIGS. 10A and 10B, respectively, are perspective and elevational views of another alternative embodiment of a prosthetic aortic heart valve.

FIG. 10C is a schematic elevational view illustrating the prosthetic aortic heart valve shown in FIGS. 10A and 10B implanted within a prosthetic aortic heart valve transcatheter dock at the location of the natural aortic valve. An explantation device is being deployed within the prosthetic aortic heart valve.

FIG. 10D is a schematic elevational view similar to FIG. 10C, illustrating expansion of the explantation device to grasp the prosthetic aortic heart valve and engagement of the electrically conductive element.

FIG. 10E is a schematic elevational view similar to FIG. 10D, illustrating the prosthetic aortic heart valve being withdrawn from the dock by the explantation device and collapsed into the explantation catheter.

### DETAILED DESCRIPTION OF ILLUSTRATIVE EMBODIMENTS

Generally, in accordance with various embodiments, an aortic valve replacement system is provided to facilitate the safe and effective removal and replacement of previously implanted prosthetic aortic heart valves with new or “replacement” prosthetic aortic heart valves. The system generally includes a prosthetic aortic heart valve dock, a prosthetic aortic heart valve, and a transcatheter valve removal or explantation system. Various unique features may be included in or for use with each component, thereby facilitating the replacement of the previously implanted prosthetic valve often years after the previous implantation and after years of use by the patient. The prosthetic aortic heart valve dock may be implanted surgically at SAVR or using a catheter at TAVR. The dock facilitates the anchoring of the first prosthetic aortic heart valve as well as its future removal and replacement. The “surgical dock” has a sewing ring and may be implanted surgically along with a prosthetic aortic heart valve constructed in accordance with one or more unique features discussed herein, and in anticipation of the eventual valve failure and transcatheter replacement, such as described herein. Various illustrative embodiments are shown in the figures and described below for exemplary purposes. Like reference numerals between the embodiments refer to like structure and function/methodology. It will also be understood that transcatheter (catheter-based) procedures are generally performed with the heart beating, while surgical procedures will be performed with the patient on a hemodynamic support system.

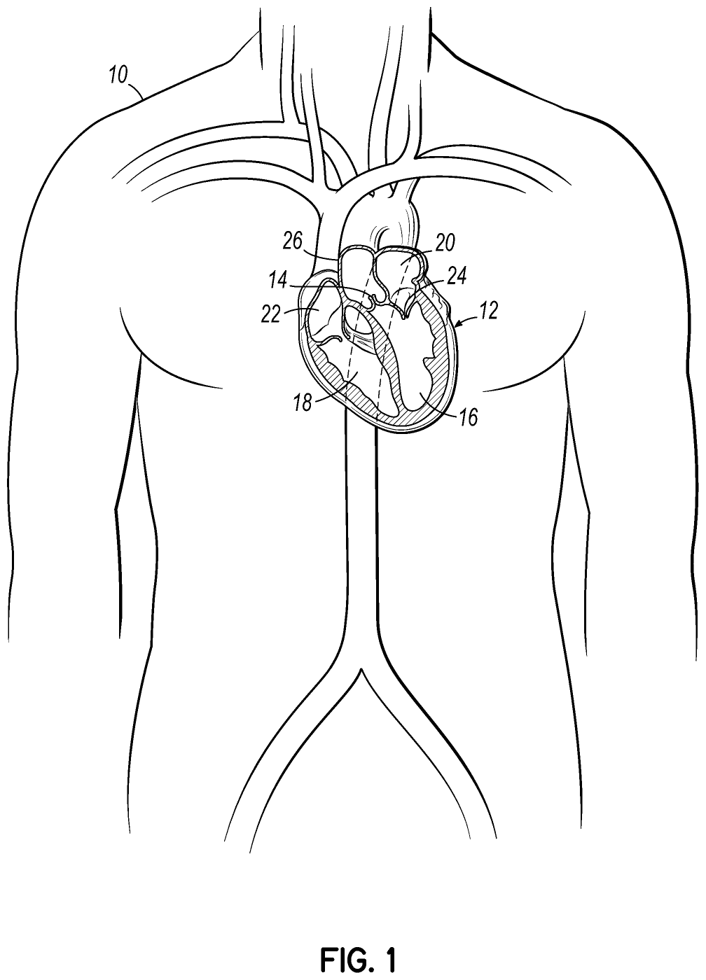

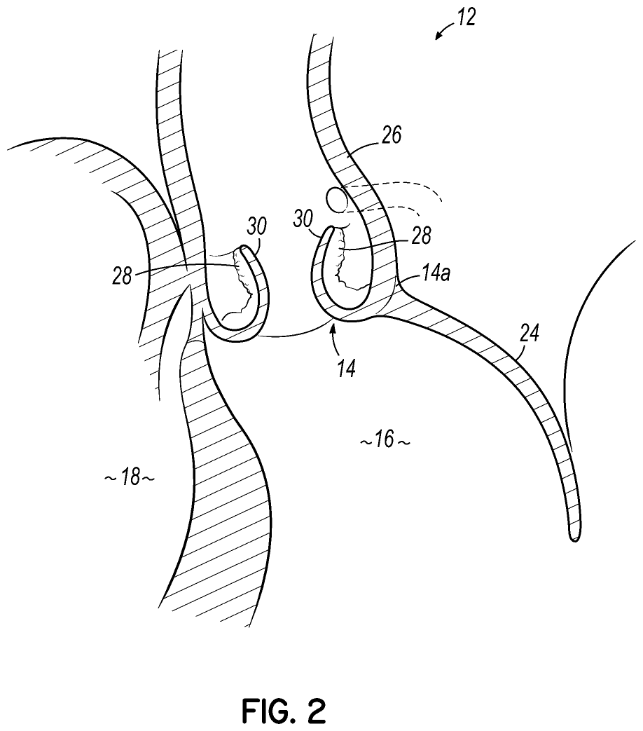

Referring to FIGS. 1 and 2, a patient 10 (FIG. 1) is shown and schematically depicts the heart 12 and the natural aortic valve 14. The heart 12, in relevant part, also includes a left ventricle 16, right ventricle 18, left atrium 20 and right atrium 22. During the diastolic phase of the heart 12, the left ventricle 16 of the heart 12 relaxes and oxygenated blood enters the left ventricle 16 from the left atrium 20 through the mitral valve 24. During the systolic phase, oxygenated blood in the left ventricle 16 is forced through the aortic valve 14 into the aorta 26. The aortic valve 14 therefore controls the vital function of allowing the transfer of oxygenated blood from the heart 12 to the rest of the body of the patient 10. This transfer is impeded if the aortic valve 14 becomes dysfunctional such as due to calcification/stenosis 28 of the valve leaflets 30 as depicted in FIG. 2. When this occurs, valve replacement may be necessary.

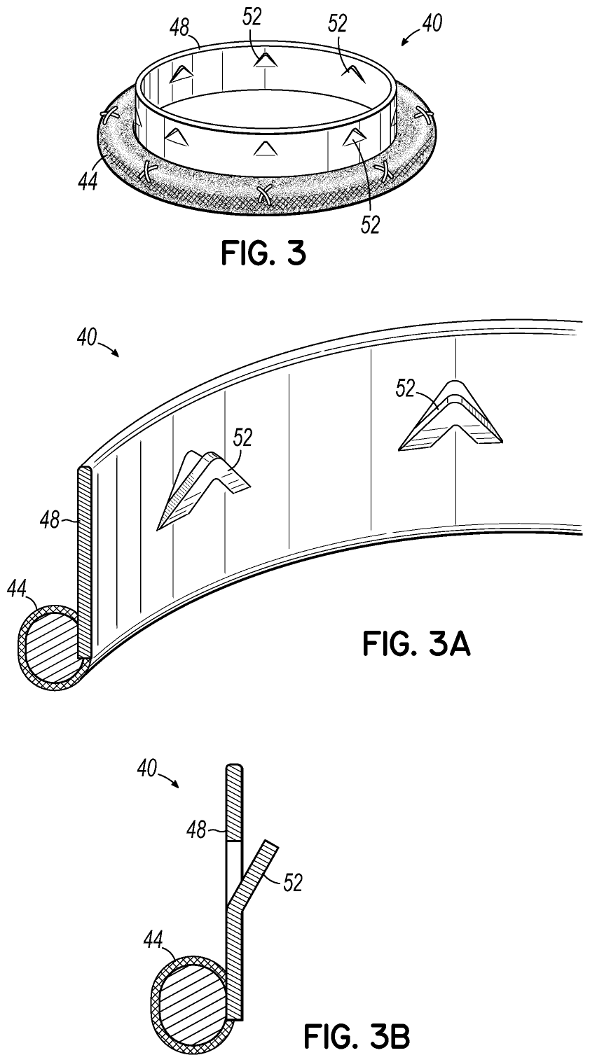

FIGS. 3, 3A, and 3B show a prosthetic aortic heart valve dock 40 constructed in accordance with an illustrative embodiment. This prosthetic aortic heart valve dock 40 is of a type that is surgically implanted and includes a sewing ring structure 44 for this purpose, which is sutured to aortic tissue proximate the natural aortic valve annulus 14a (FIG. 2). Alternatively, the prosthetic aortic heart valve dock may be a “transcatheter dock” and may even be pre-mounted on a transcatheter prosthetic aortic heart valve and implanted along with the prosthetic aortic heart valve in a single step as further discussed below in connection with an alternative, illustrative embodiment. The undeployed prosthetic aortic heart valve may be less than 6 mm in diameter (18 French). The deployed/implanted prosthetic aortic heart valve may be roughly 20-30 mm in diameter.

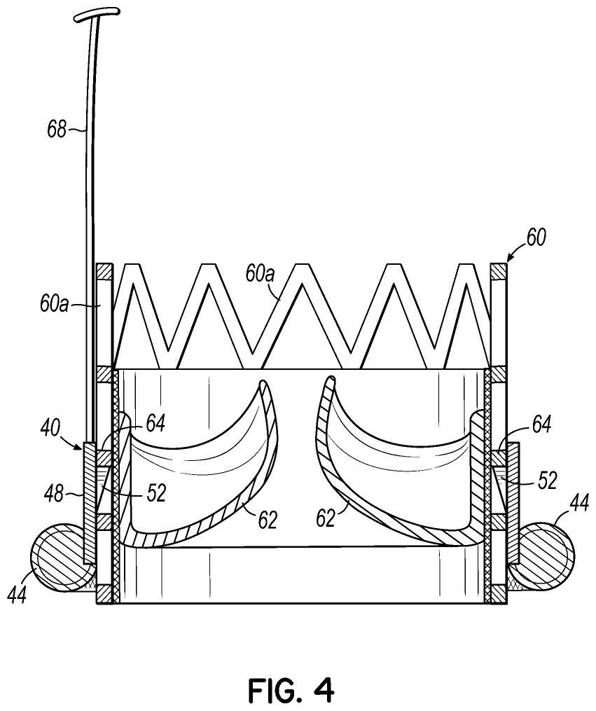

Referring again to the surgically implantable prosthetic aortic heart valve dock 40 shown in FIGS. 3, 3A, and 3B, the dock 40 may include a ring 48 of biocompatible construction, such as a biocompatible metal, for engaging with and holding an implanted prosthetic aortic heart valve as discussed further below. The fixed sewing ring 44 may be formed of suitable material for suturing to aortic tissue and promoting tissue ingrowth, such as polytetrafluoroethylene (PTFE) fabric, woven polyester fabric (Dacron), or alternatives. In accordance with this embodiment, the ring 48 includes radially inwardly directed stop elements 52. These stop elements 52 are one of many possible forms of arresting elements that may be used. In this illustrative embodiment, these stop elements 52 are “punched” V-shaped elements and act as one-way stop elements. More specifically, and as will be appreciated, these elements 52 stop the prosthetic aortic heart valve 60 (FIG. 4) from moving toward the left ventricle 16 (FIG. 1) when the prosthetic aortic heart valve 60 is engaged within the dock 40. After the prosthetic aortic heart valve 60 is implanted, the dominant (diastolic) pressure differential between the aorta 26 and the left ventricle 16 will push the prosthetic aortic heart valve 60 toward the left ventricle 16 and against the stop elements 52. When the prosthetic aortic heart valve 60 needs to be replaced, the stop elements 52 will allow movement of the prosthetic aortic heart valve 60 away from the left ventricle 16 to release the prosthetic aortic heart valve 60 during an explantation procedure. In addition to or instead of stop elements 52, the ring 48 may include frictional surface elements (not shown) that limit movement of the prosthetic aortic heart valve 60 within the dock 40. The frictional surface elements, which serve as arresting elements, may include ridges, grooves, or other surface features (not pictured) to generally limit or arrest sliding of the prosthetic aortic heart valve 60 within the prosthetic aortic heart valve dock 40.

FIG. 4 further shows an illustrative embodiment of the prosthetic aortic heart valve 60, which may be of a balloon-expandable or self-expandable type, engaged within the prosthetic aortic heart valve dock 40, with stent structure 64 stopped against the stop elements 52. This engagement prevents downward movement (as viewed in the figure) toward the left ventricle 16 (FIG. 1) but allows movement upward into the aorta 26 during an explantation procedure, to be described below. FIG. 4 further shows an electrical terminal 68 that may be engaged during the explantation procedure for purposes of facilitating the application of electrosurgical energy. While this is also described further below, such energy may be used to loosen any adhesion that occurs over time between the prosthetic aortic heart valve 60 and the dock 40 such as might occur because of bonding endothelial growth. As a preventative measure to fight endothelial growth, a drug-eluting coating or treatment may be applied to a radially outward facing surface of the prosthetic aortic heart valve 60 that engages with the prosthetic aortic heart valve dock's ring 48 and/or to a radially inward facing surface of the prosthetic aortic heart valve dock's ring 48. The coating(s) may help to prevent adhesion between these facing surfaces. Any adhesion will present a challenge to the explantation procedure and should be prevented and/or loosened to the extent possible, thereby facilitating removal of the prosthetic aortic heart valve 60 from the prosthetic aortic heart valve dock 40 when the prosthetic aortic heart valve 60 needs to be replaced.

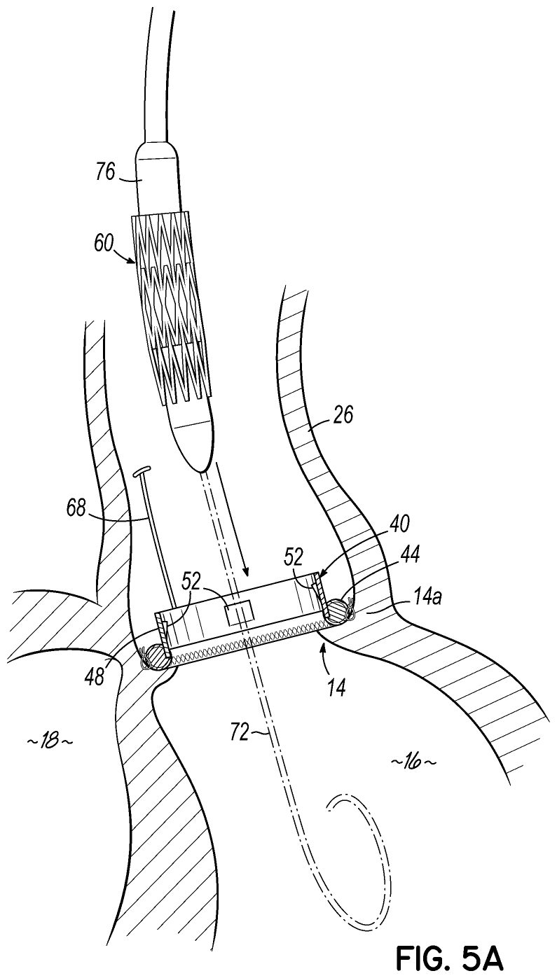

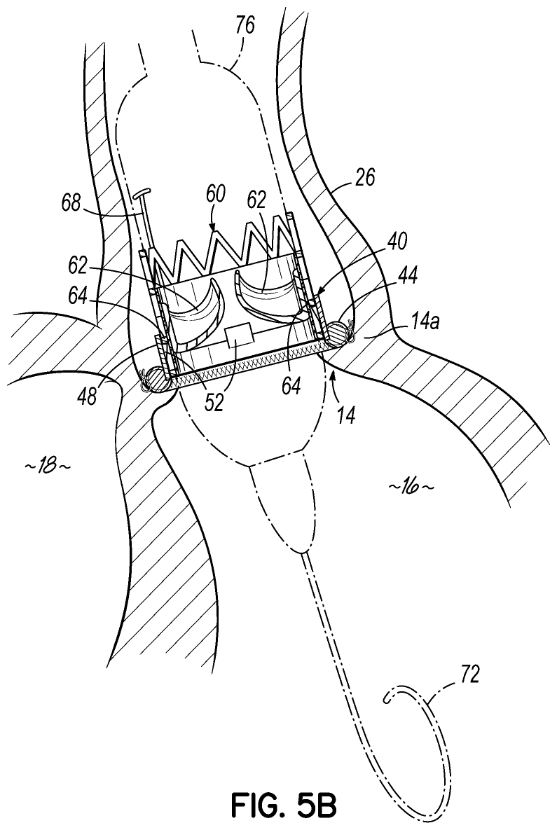

FIGS. 5A and 5B successively illustrate transcatheter implantation of a not-yet-expanded prosthetic aortic heart valve 60 along a guidewire 72. The delivery catheter has been deleted for clarity, but it will be understood that this assembly may be delivered to the previously implanted dock 40 within and through a catheter which has been directed to the surgical site within the aorta 26 (FIG. 2) along the guidewire 72. The prosthetic aortic heart valve 60 is then expanded into the position shown in FIGS. 4 and 5B, such as by using a balloon 76.

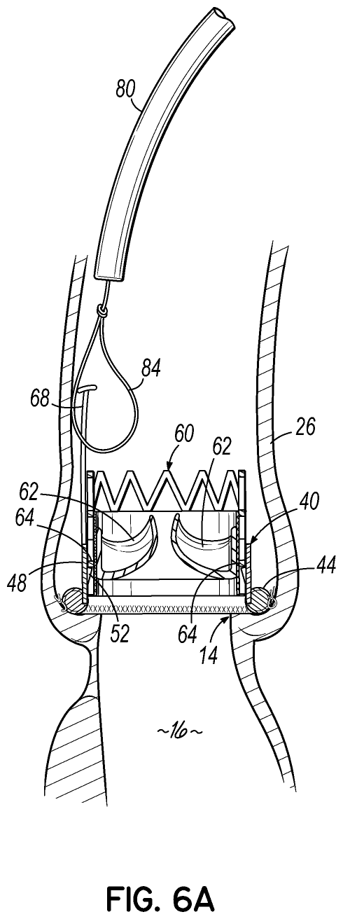

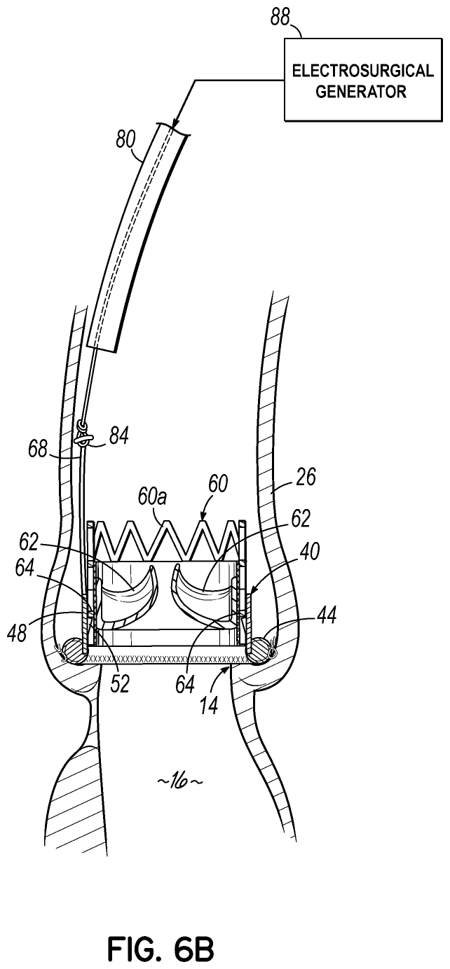

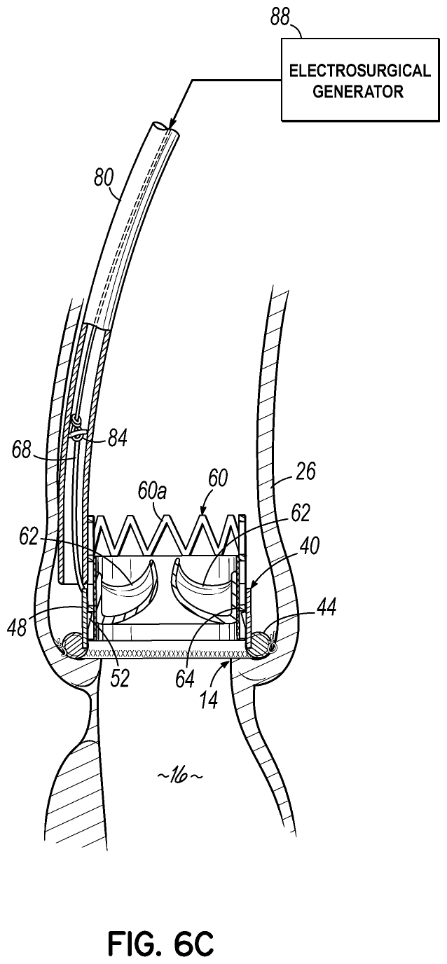

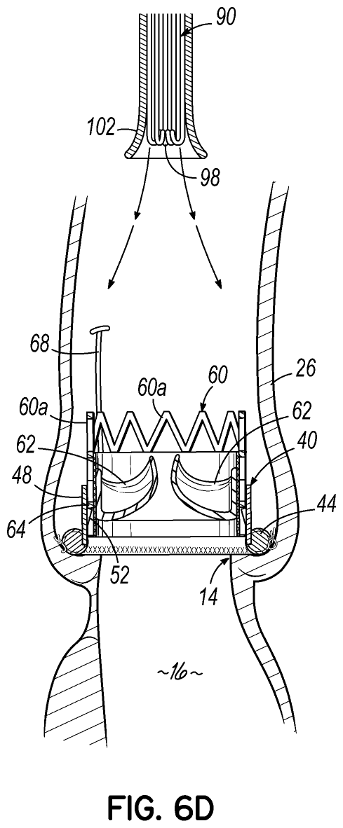

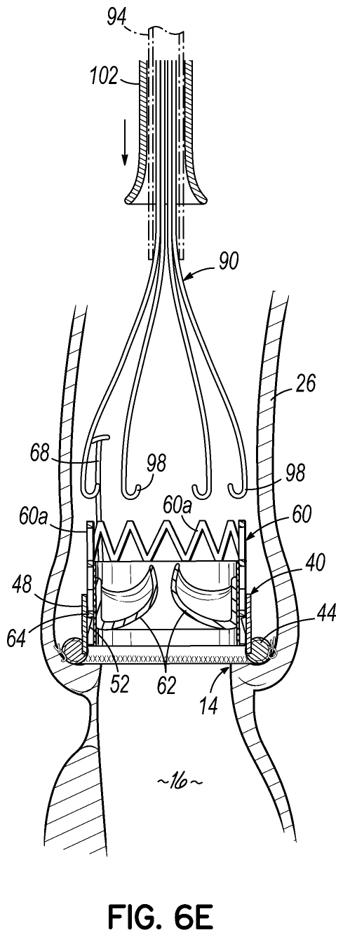

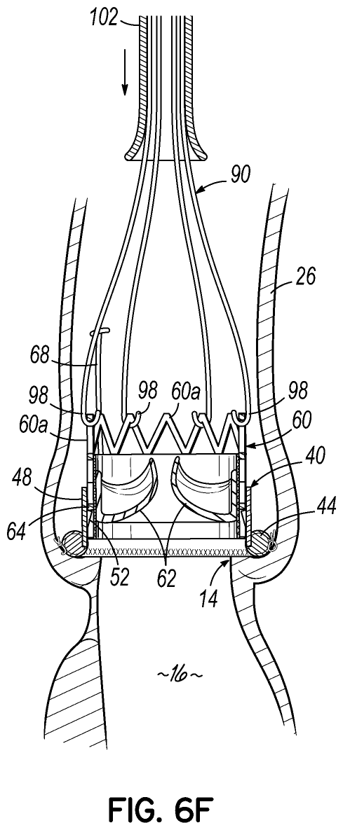

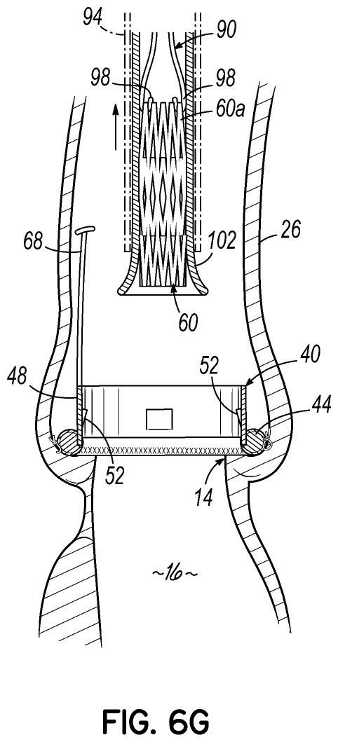

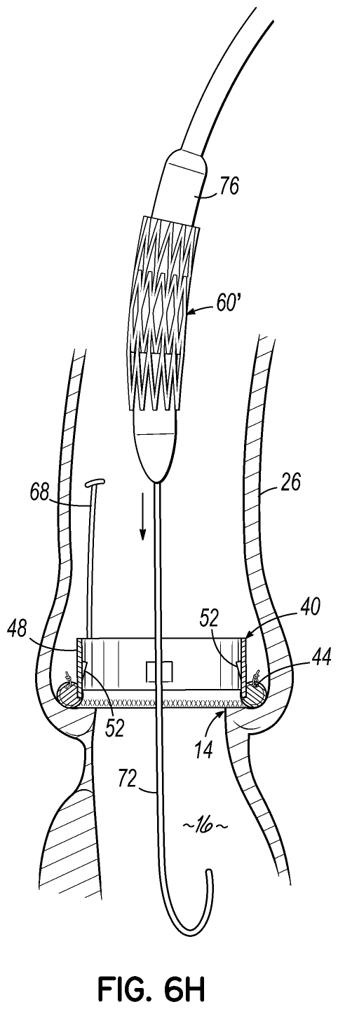

FIGS. 6A to 6H respectively illustrate the methodology or general steps involved with a transcatheter explantation procedure for an existing prosthetic aortic heart valve 60 constructed in accordance with an illustrative embodiment such as shown and described in FIGS. 4, 5A, and 5B. In FIG. 6A a catheter 80 is shown to direct a snare 84 for capturing the electrically conductive terminal 68. The terminal 68 is electrically connected to at least one of the prosthetic aortic heart valve dock 40 or the prosthetic aortic heart valve 60 and conducts electrosurgical energy, such as radiofrequency energy, to or from the metallic structure of the prosthetic aortic heart valve dock 40 and the prosthetic aortic heart valve 60 for loosening adhesion between the prosthetic aortic heart valve dock 40 and the prosthetic aortic heart valve 60. The connection of the snare 84 (or endovascular forceps or similar device) to the terminal 68 may serve as one pole in a bipolar transcatheter electrosurgical circuit. FIGS. 6B and 6C show the catheter 80 being used to insulate the electrical connection with the terminal 68 and the prosthetic aortic heart valve dock 40 from the prosthetic aortic heart valve 60, facilitating the application of electrosurgical energy from a source 88, such as a radiofrequency energy source, to the dock-valve interface with no “short-circuiting.” A catheter-based explantation device 90 is directed to the location of the prosthetic aortic heart valve 60 and prosthetic aortic heart valve dock 40 as shown in FIG. 6D. The device 90 is deployed from a collapsed condition within a catheter 94, for example, to an expanded condition as shown in FIG. 6E. Hook-like elements 98 of the device 90 engage with stent-like structure 60a of the prosthetic aortic heart valve 60 as shown in FIG. 6F. The connection of the explantation device 90 to the prosthetic aortic heart valve 60 may serve as the other pole in a bipolar transcatheter electrosurgical circuit, allowing localized application of electrosurgical energy, such as radiofrequency energy, to the interface between the prosthetic aortic heart valve 60 and the prosthetic aortic heart valve dock 40. Such application of electrosurgical energy may at least partially disrupt any bonding endothelial growth between the prosthetic aortic heart valve dock 40 and the prosthetic aortic heart valve 60, facilitating removal of the prosthetic aortic heart valve 60 from the prosthetic aortic heart valve dock 40 when the prosthetic aortic heart valve 60 needs to be replaced.

As and/or after any adhesion between the prosthetic aortic heart valve 60 and prosthetic aortic heart valve dock 40 is loosened or eliminated, as further shown in FIG. 6G, the prosthetic aortic heart valve 60 is pulled into a collapsed condition and further into an explantation catheter 102 as shown in FIG. 6G. The explantation process is completed by removing the prosthetic aortic heart valve 60 from the patient, leaving the prosthetic aortic heart valve dock 40 in place. FIG. 6H shows a subsequent step of replacing the old, explanted prosthetic aortic heart valve 60 with a new prosthetic aortic heart valve 60′ in a transcatheter implantation procedure such as shown and described in connection with FIGS. 5A and 5B.

In some embodiments, “intravascular lithotripsy” may be employed to help facilitate the explantation of the prosthetic aortic heart valve 60. Intravascular lithotripsy involves inflating an intravascular lithotripsy balloon at the treatment site and subsequently activating “emitters” within the balloon, which send acoustic pulses (“shockwaves”) out from the balloon to fracture nearby calcium without damaging adjacent soft tissue. Intravascular lithotripsy may be employed to fracture calcium on the degenerated leaflets 62 of an old prosthetic aortic heart valve 60, facilitating more complete collapsing of the prosthetic aortic heart valve 60 and its successful removal through an explantation catheter 102. Intravascular lithotripsy may be used in addition to electrosurgical separation of the prosthetic aortic heart valve 60 from the prosthetic aortic heart valve dock 40, prior to mechanical explantation of the prosthetic aortic heart valve 60. Intravascular lithotripsy may help to avoid a likely problem, where the prosthetic aortic heart valve leaflets 62 have a significant calcium buildup and are so rigid that they prevent the prosthetic aortic heart valve 60 from collapsing adequately to be removed via the explantation catheter 102.

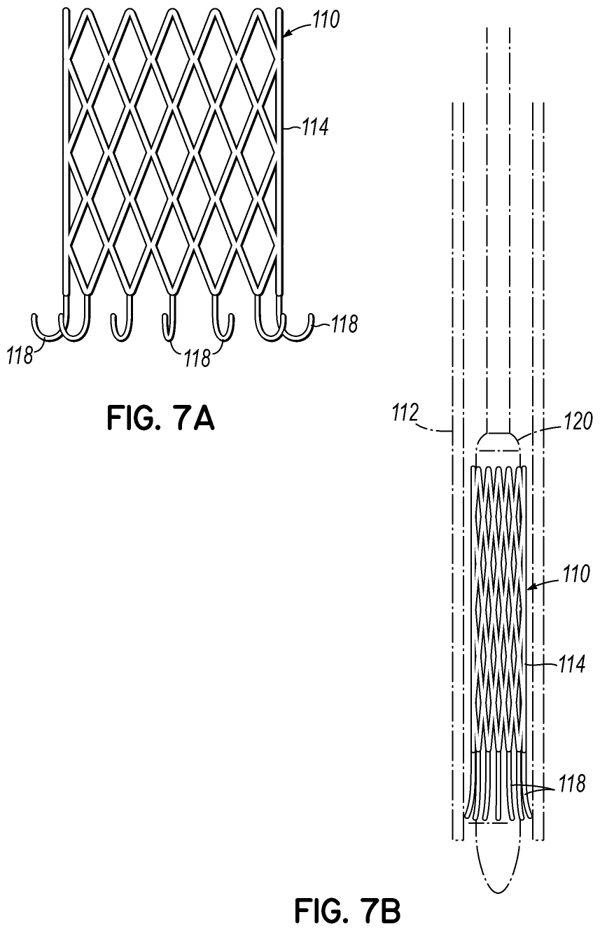

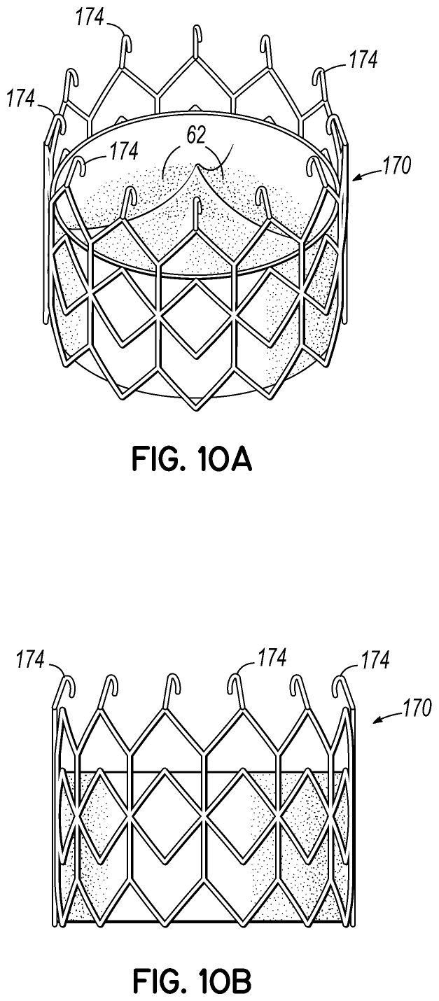

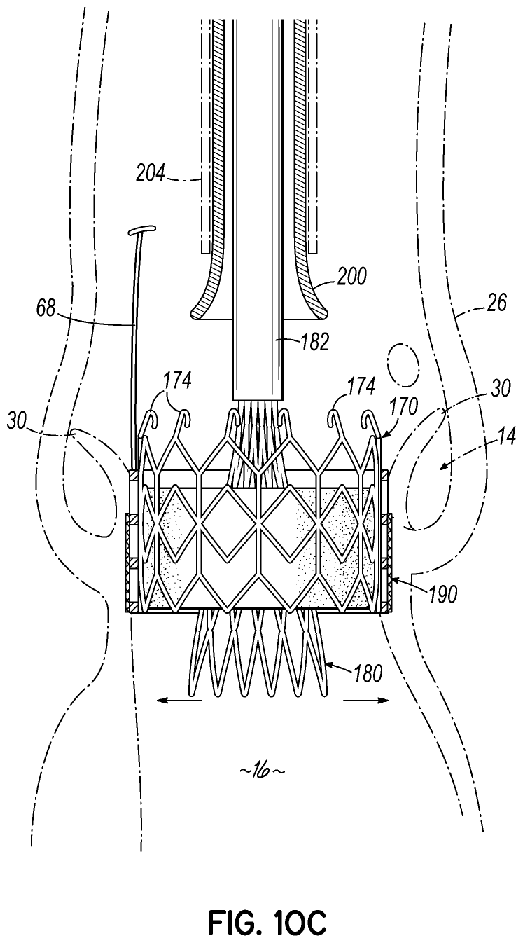

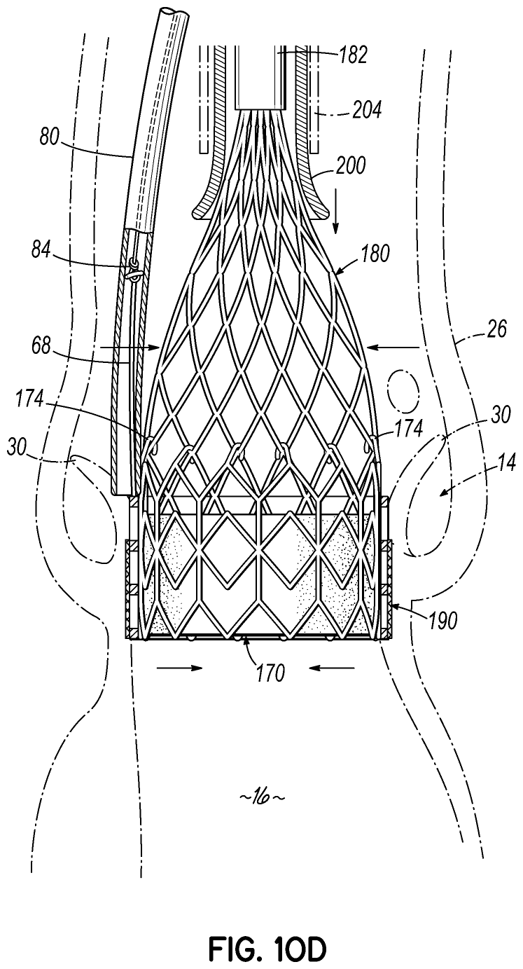

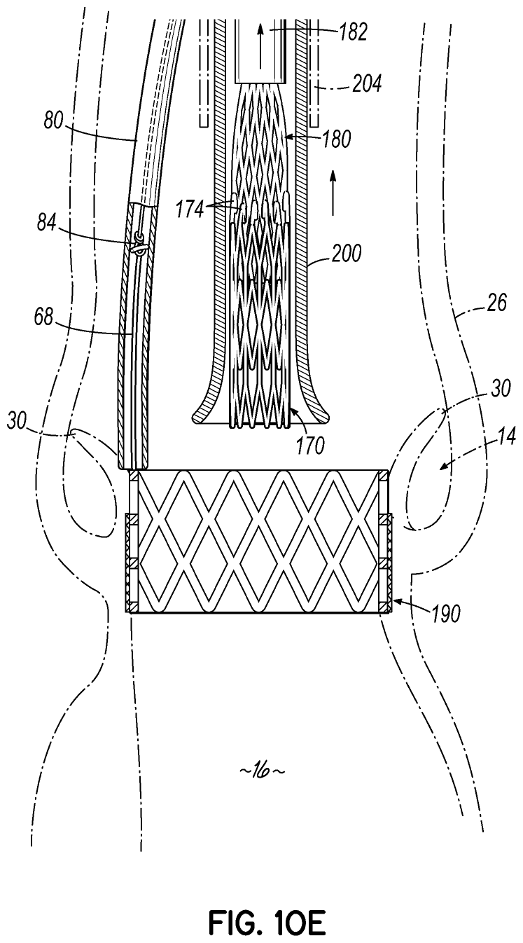

FIGS. 7A and 7B illustrate an alternative embodiment of an explantation device 110 moveable between a collapsed position within a catheter 112 (FIG. 7B) and an expanded position (FIG. 7A). In this embodiment, and unlike the previous embodiment, the device 110 includes a body 114 formed with a stent-like structure facilitating its ability to expand and contract. Hook-like elements 118 project from the distal end of the body 114. Alternatively, hook-like elements or other engagement elements may be provided on the prosthetic aortic heart valve and, in this case, the stent-like structure of the body 114, or other engagement elements may be used to engage and couple with the hook-like or other engagement elements of the prosthetic aortic heart valve. An example of this type of alternative structure is shown and described below in connection with FIGS. 10A-10E. Expansion of the explantation device 110 may take place upon extrusion or pushing the device 110 from the distal tip of a catheter, such as shown with respect to catheter 94 in FIG. 6E. That is, the explantation devices described herein may be self-expanding, as shown with respect to the device 90 in the previous embodiment. Alternatively, expansion may take place by using a balloon 120, such as shown in FIG. 7B. The material of the devices 90, 110 and other explantation devices may be, for example, designed to cause self-expansion when the device is unconstrained. A material having superelastic properties can provide this ability, with one example being nickel titanium alloy.

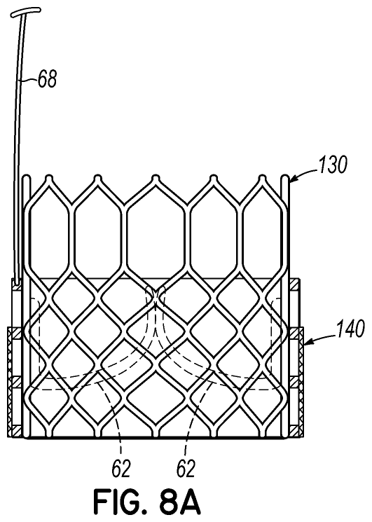

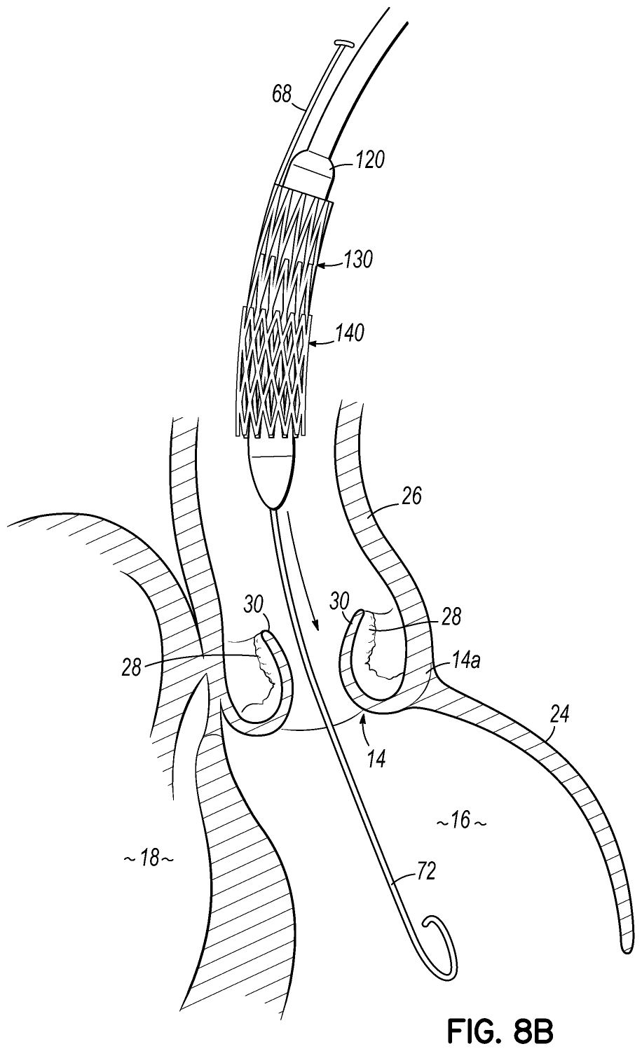

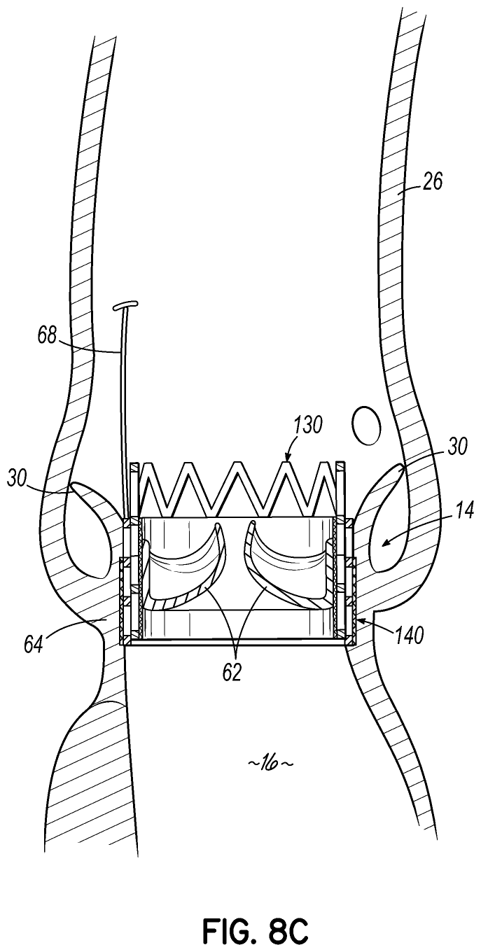

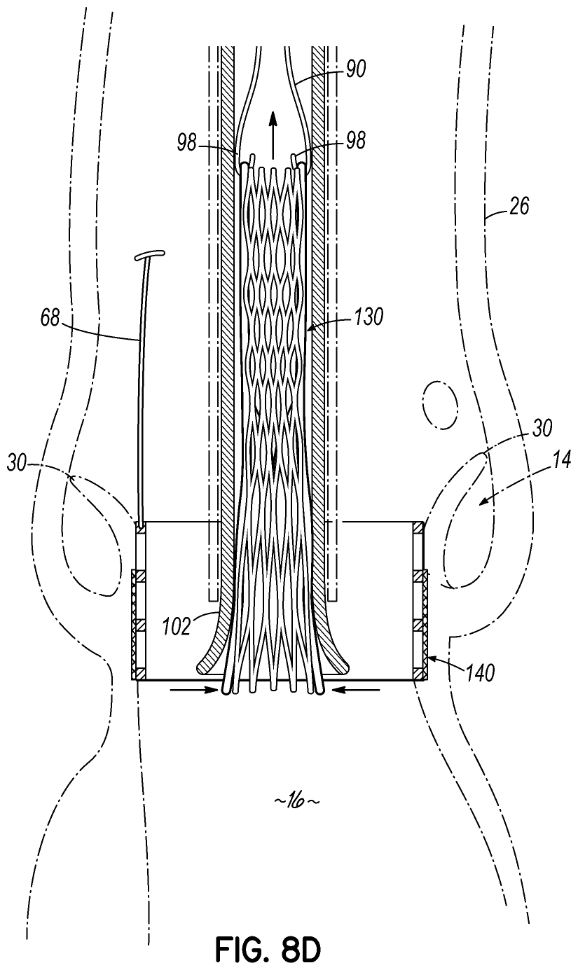

FIGS. 8A through 8D illustrate another alternative embodiment in which a prosthetic aortic heart valve 130 and a prosthetic aortic heart valve transcatheter dock 140 are simultaneously delivered and implanted during a transcatheter procedure. Although not shown, the prosthetic aortic heart valve 130 and/or prosthetic aortic heart valve transcatheter dock 140 may include one-way stop elements such as the elements 52 previously described for preventing movement into the left ventricle 16 but allowing explantation in the opposite direction. FIG. 8A shows the combination of the prosthetic aortic heart valve 130 and the transcatheter prosthetic aortic heart valve transcatheter dock 140 in an expanded condition. FIGS. 8B through 8D illustrate the process of implantation and explantation. This process occurs in a manner generally as discussed above, with the major exception being that the prosthetic aortic heart valve transcatheter dock 140 is not surgically implanted by suturing it to the aortic tissue. Instead, the transcatheter prosthetic aortic heart valve dock 140 is a collapsible and expandable stent-like structure. The prosthetic aortic heart valve transcatheter dock 140 is located and fixed on the radially outer surface of the prosthetic aortic heart valve 130 in both the not-yet-expanded condition (FIG. 8B) and the expanded condition (FIGS. 8A and 8C) at the time of implantation. As with other embodiments, the implanted prosthetic aortic heart valve 130 functions within the prosthetic aortic heart valve transcatheter dock 140, as shown in FIG. 8C, in place of the natural aortic valve 14 (FIG. 2) for as long as possible (e.g., 10-15 years). When the prosthetic aortic heart valve 130 needs to be replaced, an explantation procedure is performed, such as in accordance herewith, and the prosthetic aortic heart valve 130 is collapsed relative to the prosthetic aortic heart valve transcatheter dock 140 and removed as shown in FIG. 8D. The prosthetic aortic heart valve transcatheter dock 140 remains more or less expanded, its radially outward surface having bonded over time to adjacent aortic tissue by endothelialization. Although not shown, a new prosthetic aortic heart valve is then implanted using a transcatheter procedure such as generally shown and described in connection with FIGS. 5A and 5B.

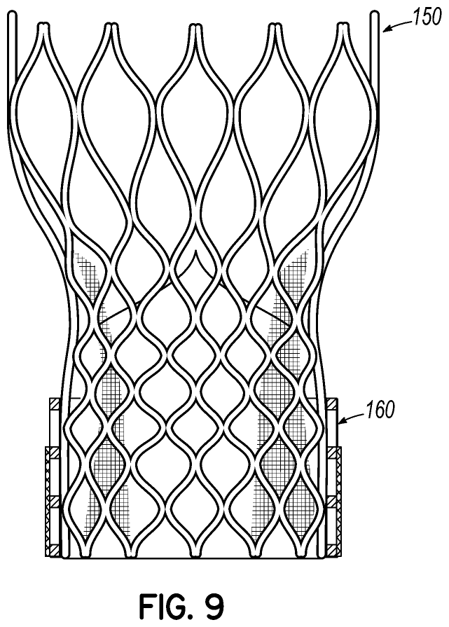

FIG. 9 illustrates another alternative embodiment of a prosthetic aortic heart valve 150 integrated with a prosthetic aortic heart valve transcatheter dock 160, both of which are implantable using a catheter (not shown), such as in a simultaneous catheter-based procedure generally described in connection with FIGS. 8B and 8C. As described in connection with FIG. 8D, the prosthetic heart valve 150 may be independently collapsed relative to the permanently expanded prosthetic aortic heart valve transcatheter dock 160 during an explantation procedure when the prosthetic aortic heart valve 150 needs to be replaced. A new prosthetic aortic heart valve may then be implanted using a transcatheter procedure.

FIGS. 10A through 10E illustrate another alternative embodiment of a prosthetic aortic heart valve 170 similar to that described in connection with FIGS. 6A through 6H. The primary difference between these embodiments is that in the embodiment of FIGS. 10A through 10E, the prosthetic heart valve 170 includes hook-like (or other) engagement elements 174, such as extending from the end located in the aorta 26 (FIG. 10C). As shown in FIGS. 10C through 10E, these hook-like elements 174 are grasped by an explantation device 180, which includes a stent-like structure, upon expansion of the device 180. The device 180 may self-expand as it is pushed or extruded from a catheter 182. As previously described in connection with other embodiments, the prosthetic heart valve 170 is then de-coupled from the prosthetic aortic heart valve dock 190, collapsed, and pulled along with the explantation device 180 into the distal end of an explantation catheter 200 and/or sheath 204. The prosthetic aortic heart valve dock 190 may be constructed in any of the manners contemplated herein and may have been previously implanted surgically or with a catheter. It will be appreciated that the procedures disclosed and otherwise contemplated may utilize many standard components and equipment that are not shown, for clarity, as such will be well understood. The prosthetic aortic heart valve transcatheter dock 190 is left secured in place and ready to receive a new prosthetic aortic heart valve (not shown), such as described hereinabove.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is Claimed is:

1. An aortic heart valve replacement system, comprising:
a prosthetic aortic heart valve movable between a collapsed condition for directing the prosthetic aortic heart valve to the location of the natural aortic heart valve and an expanded condition for fixing the prosthetic heart valve at the location of the natural aortic heart valve of a patient, wherein the collapsed condition is also used for later explanting the prosthetic heart valve, and
a prosthetic aortic heart valve dock having a ring structure configured to receive the prosthetic aortic heart valve in the expanded condition and configured to release the prosthetic aortic heart valve during an explantation procedure,
wherein at least one of the prosthetic aortic heart valve or the prosthetic aortic heart valve dock includes one or more arresting elements configured to prevent unwanted movement of the prosthetic aortic heart valve within the prosthetic aortic heart valve dock.

2. The aortic heart valve replacement system of claim 1, wherein the one or more arresting elements include at least one frictional surface element on at least one of the prosthetic aortic heart valve or the prosthetic aortic heart valve dock.

3. The aortic heart valve replacement system of claim 1, wherein the one or more arresting elements include at least one projecting portion.

4. The aortic heart valve replacement system of claim 3, wherein the projecting portion further comprises at least one projecting element directed radially inward along the prosthetic aortic heart valve dock to engage the prosthetic aortic heart valve.

5. The aortic heart valve replacement system according to claim 1, wherein the prosthetic aortic heart valve dock includes a sewing ring portion and is configured to be surgically implanted by suturing the sewing ring portion to aortic tissue at the location of the natural aortic heart valve.

6. The aortic heart valve replacement system according to claim 1, wherein the prosthetic aortic heart valve dock is configured to be implanted using a transcatheter procedure.

7. An aortic heart valve replacement system, comprising:
a prosthetic aortic heart valve movable between a collapsed condition for directing the prosthetic aortic heart valve to the location of the natural aortic heart valve and an expanded condition for fixing the prosthetic heart valve at the location of the natural aortic heart valve of a patient, wherein the collapsed condition is also used for later explanting the prosthetic aortic heart valve, and
a prosthetic aortic heart valve dock having a ring structure configured to receive the prosthetic aortic heart valve in the expanded condition and configured to release the prosthetic aortic heart valve during an explantation procedure,
wherein at least one of the prosthetic aortic heart valve or the prosthetic aortic heart valve dock includes features and/or structures that inhibit future endothelial growth and/or facilitate the destruction of endothelial growth between the prosthetic aortic heart valve and the prosthetic aortic heart valve dock at least to an extent that loosens adhesion between the prosthetic aortic heart valve and the prosthetic aortic heart valve dock.

8. The aortic heart valve replacement system of claim 7, wherein the features and/or structures for inhibiting endothelial growth further comprise a drug-eluting coating or treatment on at least one of the contacting surfaces of the prosthetic aortic heart valve or the prosthetic aortic heart valve dock.

9. The aortic heart valve replacement system of claim 8, wherein a radially outward facing surface portion of the prosthetic aortic heart valve includes the drug-eluting coating or treatment.

10. The aortic heart valve replacement system of claim 8, wherein a radially inward facing surface portion of the prosthetic aortic heart valve dock includes the drug-eluting coating or treatment.

11. The aortic heart valve replacement system according to claim 1, wherein the features and/or structures further comprise at least one electrically conductive element configured to receive a contacting conductor of an electrosurgical element to facilitate the transfer of electrical energy to the interface between the prosthetic aortic heart valve and the prosthetic aortic heart valve dock sufficient to at least assist with loosening adhesion between the prosthetic aortic heart valve and the prosthetic aortic heart valve dock.

12. The aortic heart valve replacement system of claim 11, wherein the electrically conductive element further comprises an electrical terminal extending from at least one of the prosthetic aortic heart valve or the prosthetic aortic heart valve dock and configured to be engaged by a snare, endovascular forceps, valve explantation device, or similar device.

13. An aortic heart valve replacement system, comprising:
a prosthetic aortic heart valve movable between a collapsed condition for directing the prosthetic aortic heart valve to the location of the natural aortic heart valve and an expanded condition for fixing the prosthetic aortic heart valve at the location of the natural aortic heart valve of a patient, wherein the collapsed condition is also used for later explanting the prosthetic aortic heart valve, and
a prosthetic aortic heart valve dock having a ring structure configured to receive the prosthetic aortic heart valve in the expanded condition and configured to release the prosthetic aortic heart valve during an explantation procedure when the prosthetic aortic heart valve needs to be replaced.

14. The aortic heart valve replacement system of claim 13, wherein a radially outward facing surface portion of the prosthetic aortic heart valve dock includes a coating or treatment which promotes endothelial growth.

15. The aortic heart valve replacement system according to claim 13, wherein the prosthetic aortic heart valve dock is movable between a collapsed condition and an expanded condition for implanting the prosthetic aortic heart valve dock at the location of the natural aortic heart valve of the patient.

16. An aortic heart valve replacement system, comprising:
a prosthetic aortic heart valve movable between a collapsed condition for directing the prosthetic aortic heart valve to the location of the natural aortic heart valve and an expanded condition for fixing the prosthetic heart valve at the location of the natural aortic heart valve of a patient, wherein the collapsed condition is also used for later explanting the prosthetic aortic heart valve, and
a prosthetic aortic heart valve explantation device having a portion movable between an expanded condition configured to engage the prosthetic aortic heart valve and a collapsed condition configured to be directed within a catheter,
wherein the prosthetic aortic heart valve includes engageable elements capable of being engaged by the portion of the explantation device allowing the prosthetic aortic heart valve to be pulled into the catheter.

17. The aortic heart valve replacement system of claim 16, wherein the engageable elements further comprise hook-like elements.

18. The aortic heart valve replacement system of claim 16, wherein the engageable elements are part of a radially expandable and collapsible stent structure.

19. The aortic heart valve replacement system of claim 16, wherein the explantation device further comprises hook-like elements configured to engage with the prosthetic aortic heart valve.

20. The aortic heart valve replacement system of claim 16, wherein the explantation device engages with the stent-like structure of the prosthetic aortic heart valve.

21. The aortic heart valve replacement system of claim 16, wherein the explantation device engages with the prosthetic aortic heart valve by expanding in a radially outward direction.

22. The aortic heart valve replacement system of claim 16, wherein the explantation device engages with the prosthetic aortic heart valve by collapsing in a radially inward direction.

23. The aortic heart valve replacement system of claim 16, wherein the explantation device includes a steerable catheter.

24. The aortic heart valve replacement system of claim 16, wherein the explantation device includes a catheter having a bent distal tip portion.

25. The aortic heart valve replacement system of claim 16, wherein the explantation device includes a snare-like structure configured to engage the prosthetic aortic heart valve.

26. An aortic heart valve replacement method, comprising:
directing a catheter to the location of the natural aortic heart valve where a prosthetic aortic heart valve was previously implanted,
removing the prosthetic aortic heart valve from the prosthetic aortic valve dock,
collapsing the prosthetic aortic heart valve into a distal end of the catheter,
explanting the prosthetic aortic heart valve from the patient through the catheter, and
implanting a replacement prosthetic aortic heart valve into the prosthetic aortic valve dock using a transcatheter procedure.

27. The method of claim 26, wherein the method further comprises:
releasing endothelial growth and attendant adhesion between a prosthetic aortic heart valve dock and the prosthetic aortic heart valve.

28. The method of claim 27, wherein releasing the endothelial growth includes applying electrosurgical energy to at least one of the prosthetic aortic heart valve dock or the prosthetic aortic heart valve.

29. The method of claim 26, wherein the prosthetic aortic heart valve and the replacement prosthetic aortic heart valve are each secured in the prosthetic aortic heart valve dock using one or more arresting elements configured to prevent unwanted movement of the prosthetic aortic heart valve within the prosthetic aortic heart valve dock.

30. The method of claim 26, wherein the prosthetic aortic heart valve dock is surgically implanted by suturing the sewing ring portion to aortic tissue at the location of the natural aortic heart valve.

31. The method of claim 26, wherein the prosthetic aortic heart valve dock is implanted using a transcatheter procedure.

32. The method of claim 27, wherein releasing endothelial growth between the prosthetic aortic heart valve dock and the prosthetic aortic heart valve includes establishing an electrical connection with at least one of the prosthetic aortic heart valve and the prosthetic aortic heart valve dock to facilitate the transfer of electrosurgical energy to the interface between the prosthetic aortic heart valve and the prosthetic aortic heart valve dock sufficient to at least assist with disengagement of adhesion between the prosthetic aortic heart valve and the prosthetic aortic heart valve dock.

33. The method of claim 32, wherein establishing the electrical connection further comprises engaging an electrical terminal extending from the prosthetic aortic heart valve dock, engaging an electrical terminal extending from the prosthetic aortic heart valve, and/or engaging the prosthetic aortic heart valve itself.

34. The method of claim 26, wherein removing the prosthetic aortic heart valve from the prosthetic aortic heart valve dock includes engaging engageable elements of the prosthetic aortic heart valve with an explantation device and pulling the prosthetic aortic heart valve into the catheter.

35. The method of claim 34, wherein the explantation device further comprises hook-like elements and removing the prosthetic aortic heart valve further comprises grasping the prosthetic aortic heart valve with the hook-like elements.

36. The method of claim 34, wherein the explantation device further comprises a stent-like structure and removing the prosthetic aortic heart valve further comprises engaging and holding the prosthetic aortic heart valve with the stent-like structure.

37. The method of claim 36, wherein engaging and holding the prosthetic aortic heart valve further comprises expanding the stent-like structure in a radially outward direction.

38. The method of claim 36, wherein engaging and holding the prosthetic aortic heart valve further comprises collapsing the stent-like structure in a radially inward direction.

39. The method of claim 26, wherein directing the catheter further comprises directing a steerable catheter to the location of the natural aortic heart valve where a prosthetic aortic heart valve was previously implanted.

40. The method of claim 26, wherein directing the catheter further comprises directing a catheter with a bent distal tip portion to the location of the natural aortic heart valve where a prosthetic aortic heart valve was previously implanted.

41. The method of claim 26, wherein intravascular lithotripsy is used to fracture calcium on degenerated leaflets of an old prosthetic aortic heart valve, facilitating more complete collapsing of the old prosthetic aortic heart valve and its successful removal through an explantation catheter.
